(19) United States
(12) Patent Application Publication
(10) Pub. No.: US 2002/0165611 A1
(43) Pub. Date: Nov. 7, 2002

(54) GRAFT MATERIAL CONVENIENCE PACKAGE

(76) Inventors: ROBERT-JAN ENZERINK, DAVIS, CA (US); PETER WEHRLY, BRENTWOOD, CA (US)

Correspondence Address: SUSANNE M. HOPKINS, ESQ., LIFENET, 7101 BLOOMSBURY LANE, SPOTSYLVANIA, VA, 22553, US

(21) Appl. No.: 09/461,829

(22) Filed: Dec. 15, 1999

Related U.S. Application Data
(60) Provisional application No. 60/113,312, filed on Dec. 22, 1998.

(51) Int. Cl. A61F 2/08

(52) U.S. Cl. 623/13.11; 623/13.14

(57) ABSTRACT

According to the present invention, graft material is packaged for convenient use. Packaged, sterilized, ready-to-use replacement grafts are provided in a variety of lengths with pre-attached sutures for easy insertion and may be further pre-sutured to one or two bone plugs. Thus, a surgeon may select a graft of the appropriate configuration, length, and size for use during reconstructive surgery, thereby eliminating the surgery time previously spent on harvesting and/or constructing the graft.

This application claims priority under 35 U.S.C. § 119 (e) to U.S. Provisional Application No. 60/113,312, filed Dec. 22, 1998, which is expressly incorporated by reference herein.

### FIELD OF THE INVENTION

The present invention relates to a convenient packaged ligament graft; more particularly to a sterilized preserved ready to use ligament graft; and most particularly to a convenient sterilized preserved ready to use ligament graft for replacement of a cruciate ligament. The present invention also relates to methods of preparing and using the same.

### BACKGROUND AND SUMMARY OF THE INVENTION

The prior art is replete with examples of cruciate ligament replacements. In replacing an anterior or posterior cruciate ligament, standard techniques often involve drilling bone tunnels through the tibia and femur, inserting replacement ligament material in the bone tunnels, and securing the ends. See, for example, U.S. Pat. No. 5,354,300, hereby incorporated by reference. The prior art also includes examples of replacement ligaments that have bone plugs at one or both ends. Replacement ligaments including bone plugs are particularly useful, as the bone plugs fuse into the prepared bone tunnel, healing quickly and providing a secure attachment for the replacement ligament. Replacement ligaments may be anchored in the bone tunnels by interference screw, cross-pin, tab-loop anchor, screw-and-washer, or a variety of other means. See, for example, U.S. Pat. Nos. 5,562,671 and 4,950,270, incorporated herein by reference.

Prior art replacement ligaments often involve autografts, for which suitable material is harvested from elsewhere in the patient's body. The patella tendon is widely used for such graft material, as it can be harvested with bone plugs at both ends. However, patients often experience considerable pain at the donor site following harvest of this tendon. Furthermore, an autograft patella tendon is not always available for use, particularly in revision surgery. Autograft alternatives to the patella tendon include the semitendinosis and gracilis hamstring tendons, the central quadriceps tendon, and fascia lata. See, for example, Charles H. Brown Jr. and Joseph H. Sklar, “Graft Selection, Nonpatellar Alternatives Gain Popularity,” BioMechanics, June 1998, at 21; John P. Fulkerson and Rolf Langeland, “An Alternative Cruciate Reconstruction Graft: The Central Quadriceps Tendon,” Arthroscopy: The Journal of Arthroscopic and Related Surgery, June 1995, at 252, all hereby incorporated by reference. However, these replacement ligaments cannot be harvested with bone plugs at both ends. Thus, when using a replacement ligament of this type, the graft material is often sutured to a bone plug prior to insertion in the bone tunnel. Assembly of the replacement ligament during surgery uses significant operating room time and contributes considerably to the expense of such surgery.

Allografts may be used as replacement ligaments. However, the supply of allograft patella tendons is limited. Even when patella tendons are available, additional preparation is often required prior to use. As with autografts, available allograft semitendinosis, gracilis, quadriceps, Achilles' tendons, and flexor and extensor tendons (usually from the foot) require considerable additional preparation prior to use.

According to the present invention, graft material is packaged for convenient use. Packaged, sterilized, ready-to-use replacement grafts are provided in a variety of lengths with pre-attached sutures for easy insertion and may be further pre-sutured to one or two bone plugs. Thus, a surgeon may select a graft of the appropriate configuration, length, and size for use during reconstructive surgery, thereby eliminating the surgery time previously spent on harvesting and/or constructing the graft.

In one embodiment, the replacement ligament is provided with one bone plug. In another embodiment, the replacement ligament is provided with two bone plugs, one at each end. Such pre-attached bone plugs may improve the rate of incorporation of the soft tissue into a bone tunnel, thus improving healing rates. Long strands of sutures may be attached to one or both ends, in order to facilitate insertion. The replacement ligament material may include semitendinosis, gracilis, or quadriceps tendon, or other allograft or xenograft material. Other organic materials, such as small intestine submucosa (“SIS”), collagen scaffolds, or synthetic materials may be used. See, for example, U.S. Pat. Nos. 4,902,508 and 5,711,969, hereby incorporated by reference.

According to another embodiment of this invention, replacement ligament could be provided without bone plugs. In this embodiment, the graft material may be cut to appropriate lengths and widths, looped, and sutured. This embodiment may be particularly appropriate for use with fixation techniques which do not use bone plugs, for instance in surgical situations wherein a crosspin is used to capture a looped replacement ligament.

Still another embodiment of this invention is a method of preparing a replacement graft package, including the steps of harvesting the graft material, cutting and shaping it, then assembling, preserving, and packaging the replacement graft.

The bone and tissue for use in this invention may be obtained from tissue banks, such as LifeNet, Virginia Beach, Va. The product may be preserved by freeze drying prior to packaging and reconstituted at the time of surgery. Water or a saline solution may be used for the reconstitution. Alternatively, the replacement ligament may be frozen without drying and thawed just prior to use.

In another embodiment of the invention, the replacement ligament is used to repair a damaged cruciate ligament. A sterile graft of appropriate size is selected and inserted in pre-drilled femoral and tibial holes. After insertion, the graft can be fixed within the knee using fixation devices including interference screws, cross-pins, tab-loop anchors, and screws and washers.

A final embodiment is a kit for repairing a damaged ligament comprising the pre-packaged ligament of this invention, pre-attached sutures, and a graft fixation devices including screws, cross-pins, tab-loop anchors, screws and washers, or other fixation devices.

While this invention is described for use in reconstruction of the cruciate ligaments of the knee, it should be appreciated that the invention may be practiced in other manners as well, including in the replacement of other soft tissue, especially in the replacement of other ligaments.

### BRIEF DESCRIPTION OF THE DRAWINGS

FIG. 1 is a perspective view of a replacement ligament for use in this invention, wherein a single piece of graft material is looped three times around a pair of bone plugs;

FIG. 2 is similar to FIG. 1, except showing a line drawing representing the graft material and illustrating the direction of looping;

FIG. 3 is a sectional view along the line of 3-3 of FIG. 1;

FIG. 4 is similar to FIG. 1, except the graft material comprises a single strand which is looped around each bone plug twice;

FIG. 5 is a perspective view of an end of the replacement ligament of FIG. 4 showing the sutures extending through the construct;

FIG. 6 is a sectional view along the line of 6-6 of FIG. 5;

FIG. 7 is similar to FIG. 6, except showing an alternate graft material arrangement;

FIG. 8 is an alternative embodiment of the replacement ligament for use in this invention, showing a replacement ligament without bone plugs;

FIG. 9 is a sectional view along the line of 9-9 of FIG. 8;

FIG. 10 is similar to FIG. 8, except the replacement ligament includes a looped end;

FIG. 11 is similar to FIG. 8, except the replacement ligament includes one bone plug;

FIG. 12 is a sectional view across 12-12 of FIG. 11.

FIG. 13 is similar to FIG. 12, except showing an alternate graft material arrangement;

FIG. 14 is similar to FIG. 11, except the replacement ligament includes bone plugs at both ends; and

FIG. 15 shows the graft of FIG. 8 as it is being inserted and fixed into the knee of a patient.

### DETAILED DESCRIPTION OF THE INVENTION

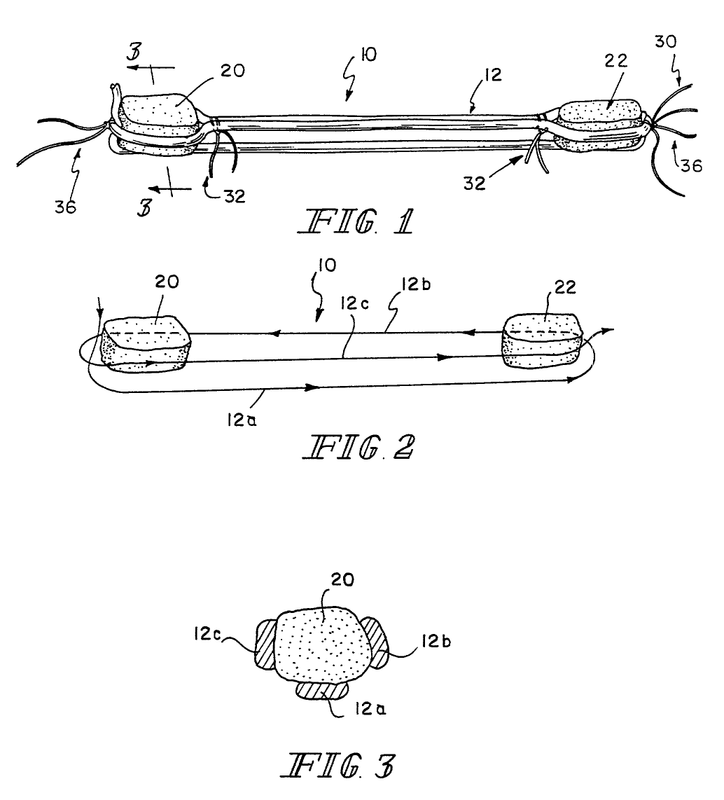

FIG. 1 shows generally a replacement ligament 10 for use in this invention. In this embodiment, replacement ligament 10 comprises graft material 12 and a set of bone plugs 20 and 22. Replacement ligament 10 of this invention also includes a set of interior sutures 32 for securing bone plugs 20 and 22 in place, and a set of end sutures 36 for use in insertion of replacement ligament 10 into a patient. Graft material 12 provides suitable flexibility and strength for ligament replacement, and bone plugs 20 and 22 are provided for bone ingrowth.

Referring still to FIG. 1, end sutures 36 may be used to secure the ends of graft material 12 in place, while interior sutures 32 secure portions of graft material 12 together to create pockets for receiving the bone plugs 20 and 22. Additionally, a long strand suture 30 may be secured to one or both ends of the replacement ligament. Such additional long strand sutures 30 may be used later to aid in insertion of replacement ligament 10 into prepared bone tunnels in the knee of the patient.

FIG. 2 illustrates a method for constructing replacement ligament 10 of FIG. 1. Graft material 12 is wrapped around first bone plug 20 to second bone plug 22, as shown by a line representing graft material 12a. Graft material 12b then loops back around second bone plug 22 and to first bone plug 20. Finally, graft material 12c wraps around first bone plug 20 and back past second bone plug 22. Thus, in this illustrative embodiment the graft material 12 is triple-stranded.

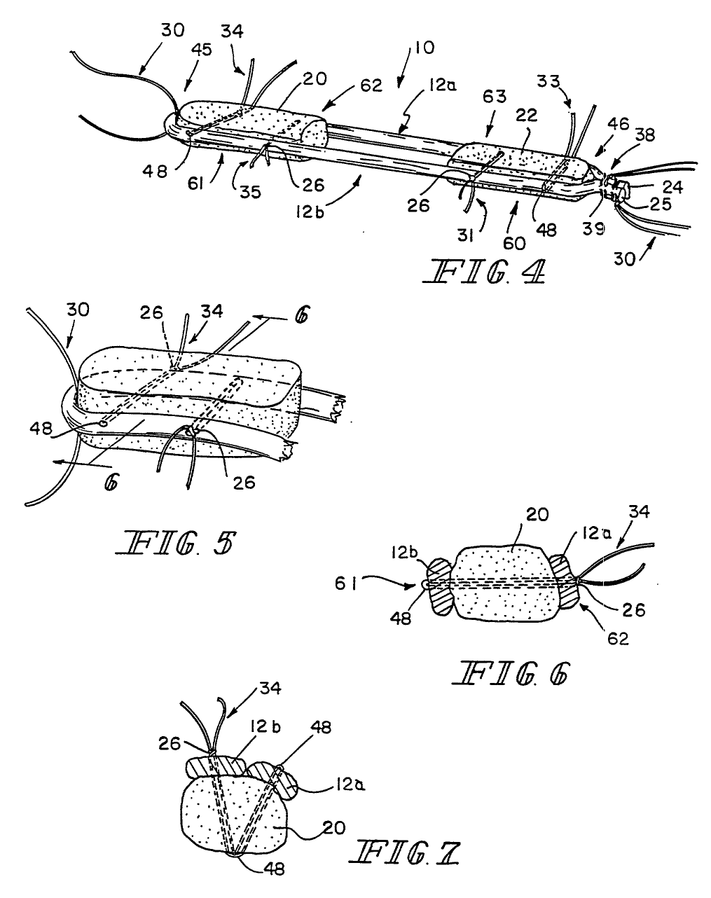

FIG. 3 shows a cross section of FIG. 1 illustrating a possible placement of graft material 12a, 12b, and 12c around bone plug 20. As illustrated in FIGS. 1 and 3, bone plugs 20 and 22 are exposed on only approximately one third of the circumference. However, in other embodiments, more surface area of the bone plugs may be exposed. This may be accomplished by looping the replacement ligament 12 twice rather than three times, as illustrated in FIGS. 4-7.

An alternative embodiment of the present invention that provides additional bone surface area is shown in FIG. 4. In this embodiment, replacement ligament 10 is provided with two bone plugs 20 and 22. Graft material 12 is double stranded. The first strand, graft material 12a, is located along a side 60 of bone plug 22, extends to bone plug 20, and is located along a side 61 of bone plug 20. Graft material 12 then loops around a proximal end 45 of bone plug 20, and the second strand, graft material 12b, is located along a second side 62 of bone plug 20, and then extends to bone plug 22 at a side 63. As illustrated, bone plug 20 is then secured to graft material 12 by a set of sutures 34 and 35. Suture 34 extends from side 62 of bone plug 20 and passes completely through bone plug 20 to side 61 where optionally a stitch 48 is made. Suture 34 then returns to side 62. As shown, suture 35, is provided in the opposite orientation and extends from side 61 of bone plug 20 and passes completely through to side 62. Suture 35 then passes back through bone plug 20 to side 61 where a knot 26 is tied. As described above, graft material 12 is also wrapped around bone plug 22. Bone plug 22 is then secured to replacement ligament 12 by sutures 31 and 33 that pass completely through bone plug 22 in the same manner as sutures 34 and 35. A set of bundle sutures 38, preferably provided in a whip stitch 39, may be used to secure ends 24 and 25 of graft material 12 beyond bone plug 22 at a distal end 46 of replacement ligament 10. Additional long strand sutures 30 may be secured to the proximal and distal ends 45 and 46 of the replacement ligament 10. It will be understood that the terms proximal and distal are used for convenience and represent the preferred orientation of replacement ligament 10 for use in anterior cruciate ligament repair. Alternative graft orientations may be required for particular applications or surgeon preference.

FIGS. 5 and 6 further illustrate a construction in which graft material 12 sutured to bone plug 20 of FIG. 4. As best seen in FIG. 5, suture 34 passes completely through bone plug 20 twice, first passing from side 62 through bone plug 20 to side 61. Preferably a stitch 48 is then made on side 61. The suture then passes back through bone plug 20 to side 62. The ends of suture 34 are then tied in a knot 26, thus securing the opposing strands 12a and 12b of graft material 12. In the embodiments of FIGS. 4-6, it is preferred that all strands of graft material 12 be secured to bone plug 20. However, for some applications surgeons may prefer replacement ligaments for which the bone plugs are provided in pockets, rather than rigidly sutured to the graft material.

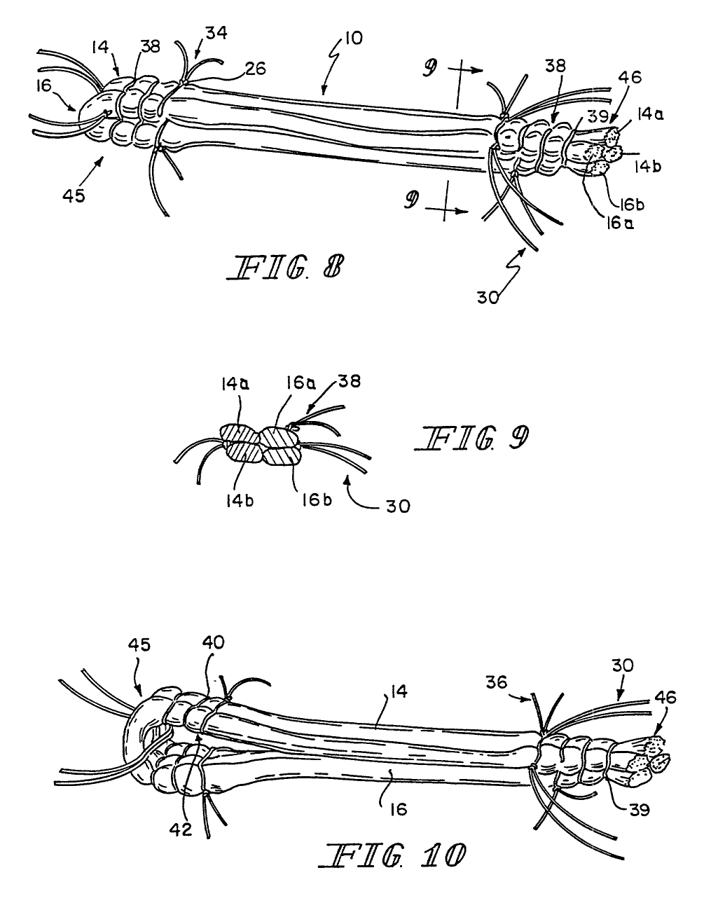

In some fixation methods, it would be preferable to have bone exposed on both sides of the graft, as shown in FIGS. 4-6. With other fixation methods, it may be preferable to have the greatest possible exposed surface area of bone plug 20 on one side, as seen in FIG. 7. FIG. 7 shows an alternative graft arrangement in which the graft strands 12a and 12b are placed together on one side of bone plug 20 and secured by suture 34. Also, the replacement ligament may be provided in a straight single length and sutured to bone plugs without looping at all. Finally, by varying the size of the bone plugs in relation to the replacement ligament width, more of the circumference of the bone plug may be exposed without sacrificing necessary strength of the replacement ligament.

Another embodiment is illustrated in FIG. 8. In this embodiment, a replacement ligament 10 is provided without bone plugs. Graft material 12 may be single-stranded or multiple-stranded. If graft material 12 is multiple-stranded as shown, the multiple strands may be created by bundling individual strands, by folding a strand back upon itself, or by a combination of both. FIG. 9 shows a cross section of FIG. 8 illustrating the multiple-stranded bundle created by the individual strands 14 and 16 folded back upon themselves and secured by bundle sutures 38.

Still referring to FIG. 8, multiple stranded replacement ligament graft distal end 46 maybe secured using bundle sutures 38, preferably provided in a whip stitch 39. As shown, 14a, 14b, 16a, and 16b are secured with bundle sutures 38 in a whip stitch 39 at both proximal and 45 and distal end 46. Also as shown, long strand sutures 30 are provided as separate sutures. Alternatively, long strand sutures 30 may be provided as the ends of bundle sutures 38. For some applications, long strand sutures 30 may be omitted. In the illustrative embodiment of FIGS. 8 and 9, the replacement ligament 10 is made of two individual strands 14 and 16, which are folded back upon themselves at proximal end 45, creating a quadruple-strand graft. However, it will be understood that other strand arrangements are within the scope of this invention.

FIG. 10 shows an embodiment similar to that illustrated in FIG. 8. However, at proximal end 45, a set of semi-bundle sutures 40 only partially bundle graft material 12, creating a loop 42. Loop 42 may be particularly useful for fixation methods which employ a cross pin. Alternatively, loop 42 may be provided in applications where autograft bone plugs are preferred. A similar loop may be provided at distal end 46.

FIG. 11 illustrates an embodiment of this invention which was prepared in a manner similar to the embodiment illustrated in FIG. 8, but includes one bone plug 20. In this illustrative embodiment, bone plug 20 is included within the whip stitching 39 of bundle sutures 38 at distal end 46 of replacement ligament 10. The whip stitching 39 of bundle sutures 38 may provide enough support to hold the bone plug 20 in place. Alternatively, bone plug sutures similar to those shown in FIG. 5 may be necessary to provide proper attachment strength. Other arrangements for bone plug 20 are possible within the scope of this invention. Also, the proximal end 45 of this embodiment may be looped as in FIG. 10, or closed as in FIG. 8.

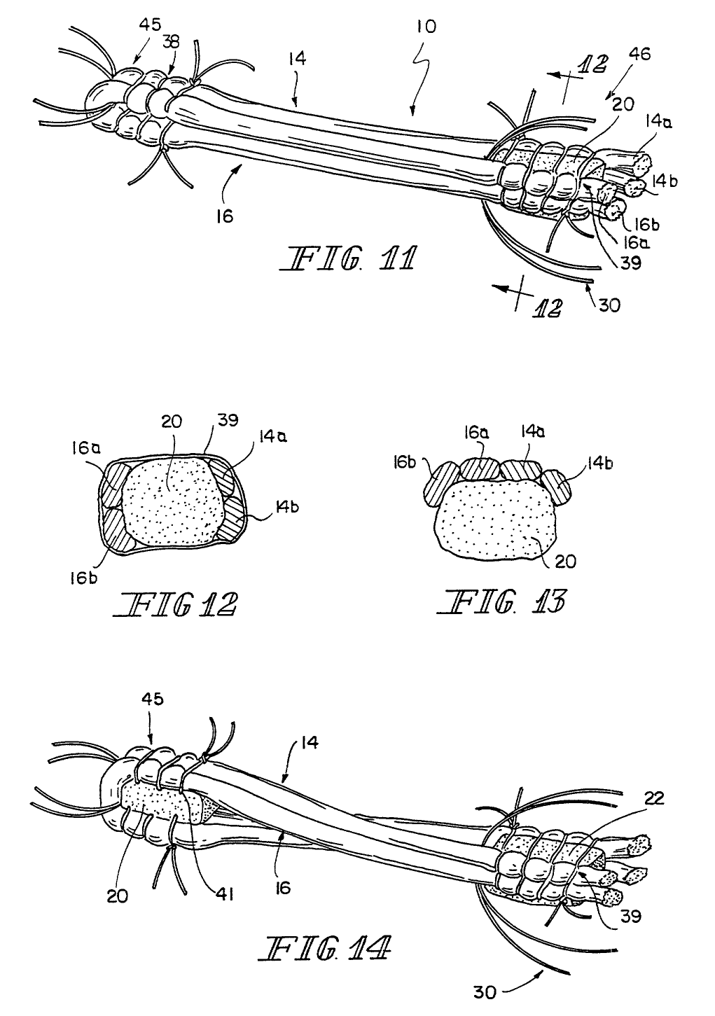

A cross section of FIG. 11, shown in FIG. 12, illustrates a possible placement of replacement ligaments 14 and 16 around bone plug 20. The whip stitching 39 of the bundle sutures 38 secures bone plug 20 within the replacement ligament strands 14a, 14b, 16a, and 16b. FIG. 13 shows a cross section of an alternative arrangement to FIG. 12 wherein all four strands of ligaments 14 and 16 may be located on one side of bone plug 20. The stitching of the bundle sutures is not shown. Alternatively, ligaments 14 and 16 may be secured with sutures similar to those shown in FIG. 7.

As illustrated in FIGS. 11-13, the graft material is provided in the quadruple strand arrangement of FIG. 8. However, other multiple strand arrangements may be suitable.

FIG. 14 illustrates another embodiment which is similar to that illustrated in FIG. 11, as the replacement ligaments 14 and 16 are looped at proximal end 45 and bone plug 22 is incorporated within the whip stitching 39 of bundle sutures 38. However, with the embodiment illustrated in FIG. 14, bone plugs 20 and 22 are provided on both ends. In this embodiment, the replacement ligaments 14 and 16 of proximal end 45 are held together with a set of bundle sutures 41. As shown, the same sutures 41 are used to suture bone plug 20. This embodiment ensures that bone plug 22 will not move during insertion into a patient's knee. Other methods of securing bone plug 20 are possible.

According to another embodiment, allograft replacement ligaments may be prepared using patella tendons which are harvested with bone plugs (not shown). Preparation of these replacement ligaments includes pre-attached sutures to facilitate subsequent insertion. The sutures may be made of absorbable or non-absorbable suture material. These replacement ligaments are provided in a variety of sizes and are packaged and sterilized for convenient use. The package may also include graft fixation devices.

In a method of construction of this invention, graft material may be obtained from a variety of sources including allograft, xenograft, or synthetic material. If allograft or xenograft material is used, it can be prepared by removing extraneous tissue, cutting the material to the proper size, and assembling into ready to use configurations, as described above. If bone plugs are provided, they are cut to the proper size, and they may be provided with slots to aid in suturing during assembly. The bone plugs may be allograft or xenograft bone material, or they may be made from bone substitutes such as hydroxyapatite, tri-calcium phosphate, or others.

Prior to assembly, the replacement ligament material may go through a series of washes. Such washes may include ALLOWASH™ solution, sterile water, isopropyl alcohol, antibiotic solution, and a final rinse with sterile water. ALLOWASH™ as described in U.S. Pat. No. 5,820,581, hereby incorporated by reference, as a solution comprising three detergents, i.e., (1) polyoxyethylene-4-lauryl ether having the chemical formula C₄H₁₉(OCH₂CH₂)₄OH, (2) octylphenol-ethyleneoxide, and (3) poly(ethylene glycol)p-nonyl-phenol-ether. The '581 patent also discloses various alternative cleaning solutions and washing protocols which may be used in the practice of this invention. The antibiotic solution may be a solution of endotoxin-free deionized/distilled water or ethanol, containing antibiotics, antiviral agents, hydrogen peroxide, permeation enhancers, organic acids, or a dilute solution of strong acids. Preferably, the antibiotic solution contains a mixture of bacitracin and polymyxin in sterile water. U.S. Pat. No. 5,797,871, hereby incorporated by reference, describes other methods of cleaning allograft bone. It should be understood that these and other techniques for cleaning the allograft tissue are within the scope of this invention.

Following assembly, the graft material may be placed under tension. For example, a tension spring may be used. This tension keeps the replacement ligament in proper alignment during subsequent preservation and packaging. The spring or other tension device may be removed before packaging or just prior to use.

Once the allograft or xenograft replacement ligaments have been washed, they are preserved. In one preferred embodiment of preparing the graft material, preservation is accomplished by a lyophilization cycle which may, for instance, last five days. Preferably, the replacement ligament is placed in sterile gauze within a sterile bottle prior to lyophilization, and the bottle is stoppered under vacuum following lyophilization to finish the packaging. When used, the replacement ligament may be reconstituted in either sterile water or saline, and such reconstitution may take approximately one hour under vacuum. For grafts which have been bottled under vacuum, this reconstitution may take place simply by introducing water or saline into the bottle by use of a sterile needle. Preferably, the needle would pierce the stopper without breaking the vacuum.

Alternatively, following washing, the replacement ligaments may be preserved by being fresh frozen. In this method of preservation, the replacement ligaments preferably are placed in a peelable soft package. Air is removed from the package by suction, and the package can be heat-sealed. A second, slightly larger, soft package may be used for additional protection of the graft material. For further sterilization, the replacement ligaments may be irradiated as is known in the art, for example with gamma radiation. Finally, the replacement ligaments are stored frozen, for instance from about −70° C. to about −80° C., and may be distributed on dry ice. When needed, frozen grafts are thawed, preferably in sterile saline. Because fresh frozen replacement ligaments are not fully dried, the reconstitution step required for lyophilized grafts may be omitted.

Various fixation devices, including interference screw, cross-pin, tab-loop anchor, screw-and-washer fixation devices, may be packaged with the replacement ligament, thus providing the surgeon with a kit for replacing a damaged ligament.

Synthetic materials may also be used in the practice of this invention. The graft material and/or bone plugs may be made of synthetic materials, and the replacement ligament may be constructed using a similar variety of techniques. The replacement ligament of this embodiment is then sterilized, and it is packaged according to the requirements of the materials used. As with allograft or xenograft replacement ligaments, the synthetic replacement ligaments may be packaged with or without graft fixation devices.

The replacement ligaments of this invention may be provided in a variety of lengths, with or without a variety of sizes of bone plugs. For ACL or PCL repair, lengths of 60 to 150 mm, preferably 90 to 100 mm may be used, with bone plugs preferably having a diameter of 6 to 10 mm. However, certain PCL techniques may require longer grafts, and individual patients or alternative fixation techniques may require grafts of larger or smaller sizes. Also, grafts for use in the replacement of other ligaments or other soft tissue may be larger or smaller. Thus, while sizes are disclosed, it will be understood that these sizes are merely typical of sizes needed for repair of the cruciate ligaments. Other sized grafts are within the scope of this invention.

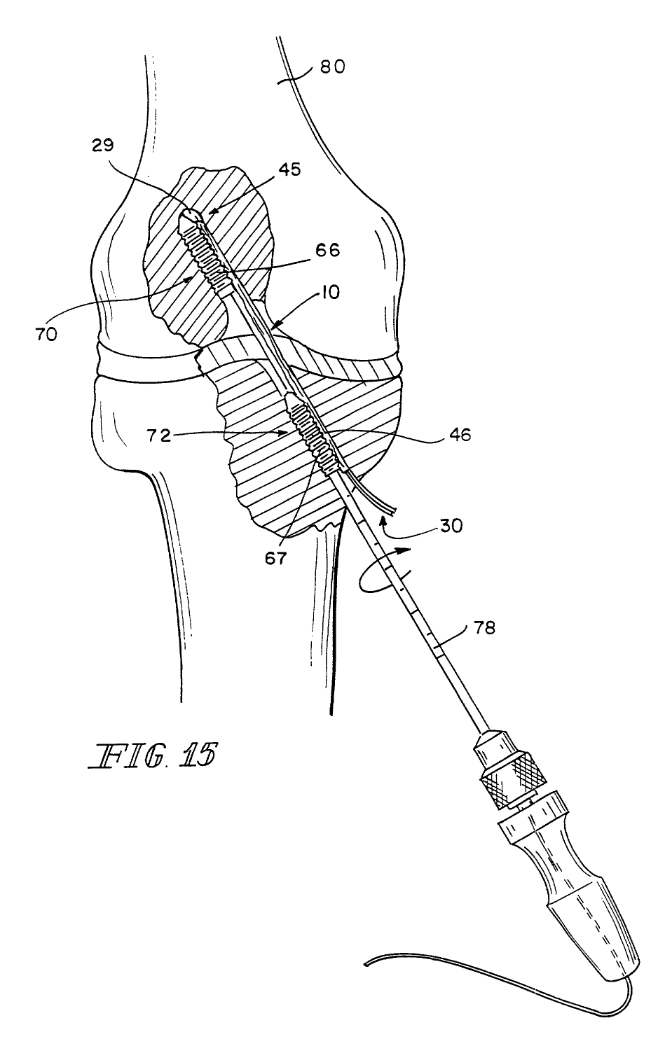

As an example of use, a replacement ligament 10 of this invention may be used to repair an anterior cruciate ligament. FIG. 15 illustrates the graft of the present invention after it has been inserted into the patient. The surgical site and the tibial tunnel 72 and femoral tunnel 70 are drilled as known in the art, see, for example, U.S. Pat. No. 5,671,695, incorporated herein by reference, preferably while replacement ligament graft 10 is reconstituting or thawing. After tibial and femoral tunnels 72 and 70, respectively, have been drilled, a slotted guide pin (not shown) may then be passed through tibial tunnel 72 and into femoral tunnel 70. The knee is placed in hyperflexion and the guide pin can be passed out the anterolateral femoral cortex, where it then exits the skin.

Distal end long strand sutures 29 of the replacement ligament 10 are looped through the slotted guide pin eyelet (not shown) and pulled through the joint, exiting at the anterolateral femur. A pin puller, for example the DePuy Pin Puller (Cat. No. 2972-96-000), may be helpful in pulling the sutures through the joint. The graft is gently pulled into the tibial tunnel 72. A guidewire is then passed through a stab wound made at the level of the tibial plateau and just medial to the patellar tendon and into the femoral tunnel 70 while maintaining a position along the femoral wall. At this point the knee is flexed an additional 15-20 degrees beyond the flexion angle used to drill the femoral tunnel 70 to accommodate the guidewire position. A fixation device 66, such as the DePuy Phantom SofThread Screw, can be used to fix replacement ligament 10 into femoral tunnel 70.

After the guidewire has engaged the proximal aspect of the femoral tunnel 70, it is tapped into femur 80 with a mallet, for example the DePuy Mallet (Cat. No. 2972-23-000), to avoid rotation of the guidewire and the fixation device. A second guidewire is then passed through the tibial tunnel 72 anterior to graft 10. It may be useful to pull graft 10 back out of the tibial tunnel 72 to introduce the tibial wire.

After both wires are in position, the graft is then pulled up the tibial tunnel 72 and into the femoral tunnel 70. The proximal long strand sutures 30 are used to determine complete seating of graft 10 in femoral tunnel 70. Once the graft is tensioned both proximally and distally, the fixation device 66 is inserted through an anteromedial puncture wound.

The fixation device 66 is then advanced until the entire head is within femoral tunnel 70 and preferably countersunk beneath an edge of the femoral tunnel 70. The knee is then placed at approximately 20-30 degrees of flexion. Graft 10 is loaded to 20 lbs. 20 times by pulling on the long strand sutures 30 exiting the tibial tunnel 72. Using approximately 15 lbs. of preload, a tibial fixation device, for example tibial screw 67, is inserted along the anterior graft surface and advanced with driver 78 until it is completely within the tibial tunnel 72.

The knee is checked for mobility and stability and when acceptable, the proximal sutures 30 are pulled through the skin and removed. Sutures and any excess graft material flush with the tibial tunnel 72 should be transected. However, care should be taken to not cut the sutures that hold the graft together. The wounds are then closed in a standard fashion. It is understood that the above is illustrative of a surgical technique employing replacement ligaments of this invention. Other surgical techniques are within the scope of this invention.

Although the invention has been described with reference to certain preferred embodiments, variations and modifications exist within the scope of the present invention.

1. A replacement package for the repair of a damaged ligament comprising:
a graft comprising a graft material having a proximal end and a distal end, a first set of sutures attached to the proximal end, and a second set of sutures attached to the distal end,
wherein the graft is preserved and provided in sterile packaging.

2. The graft package of claim 1 wherein the graft material is selected from the group consisting of allograft, xenograft, and synthetic material.

3. The graft package of claim 2 wherein the graft material is allograft and selected from the group consisting of patellar tendon, semitendinosis tendon, gracilis tendon, quadriceps tendon, Achilles' tendons, flexor tendons, extensor tendons, or fascia lata.

4. The graft package of claim 1 wherein the graft material comprises a bundle of strands, the first set of sutures securing the proximal end together, and the second set of sutures securing the distal end together.

5. The graft package of claim 1 wherein the first and second set of sutures comprise long strand sutures for aiding in subsequent placement of the graft into a patient.

6. The graft package of claim 1 wherein the first and second set of sutures each comprise whip stitch sutures, bundle sutures, and long strand sutures.

7. The graft package of claim 1 wherein the graft is provided with a bone plug secured to the proximal end.

8. The graft package of claim 7 wherein the first set of sutures secure the bone plug to the proximal end.

9. The graft package of claim 8 wherein portions of the first set of sutures extend completely through the bone plug to secure the bone plug to the graft material.

10. The graft package of claim 7 further comprising a second bone plug secured to the distal end of the replacement ligament

11. The graft package of claim 10 wherein the bone plugs are selected from a group consisting of allograft, xenograft, and synthetic material.

12. A prepackaged sterile replacement ligament comprising:
a graft comprising a bundle of graft material strands, the bundle having a proximal end and a distal end, a first set of sutures securing a first bone plug to the proximal end, a second set of sutures securing a second bone plug secured to the distal end,
wherein the graft is preserved and provided in sterile packaging.

13. The replacement ligament of claim 12 wherein the bundle is formed by at least one strand looped back on itself.

14. The replacement ligament of claim 12 wherein the graft is preserved by freezing.

15. The replacement ligament of claim 12 wherein the graft is preserved by lyophilization.

16. A method of preparing a convenient replacement graft package for use in repairing a damaged ligament, the method comprising the steps of
harvesting a piece of graft material and removing extraneous tissue;
washing the harvested piece of graft material;
cutting and shaping the piece of graft material to proper size, defining a proximal end and a distal end;
assembling the replacement graft by attaching a first set of sutures to the proximal end and attaching a second set of sutures to the distal end;
preserving the replacement graft; and
packaging the replacement graft in a sterile container.

17. The method of claim 16 wherein the assembling step further comprises attaching a first bone plug to the proximal end of the piece graft material.

18. The method of claim 17 wherein the assembling step further comprises attaching a second bone plug to the distal end of the graft material.

19. The method of claim 16 wherein the cutting and shaping step further comprises looping the piece of graft material back upon itself.

20. The method of claim 16 wherein the washing step includes using a solution comprising ALLOWASH™ solution, isopropyl alcohol, and antibiotic solution.

21. The solution of claim 20 wherein the antibiotic solution comprises at least one of the group consisting of antibiotics, antiviral agents, hydrogen peroxide, permeation enhancers, organic acids, and dilute solutions of strong acids.

22. The solution of claim 20 wherein the antibiotic solution comprises a mixture of bacitracin and polymyxin.

23. The method of claim 16 wherein the packaging step includes placing the graft in a bottle, the preserving step includes lyophilization and the graft is placed in the bottle prior to lyophilization.

24. The method of claim 23 wherein the packaging step further comprises stoppering the bottle under vacuum following lyophilization.

25. The method of claim 16 wherein the preserving step comprises freezing and the packaging step comprises placing the graft in a peelable soft package, removing air by suction, heat-sealing the package, and freezing the package.

26. The method of claim 25 wherein the packaging step further comprises placing the peelable soft package into a second larger package.

27. The method of claim 25 wherein the package is irradiated prior to freezing.

28. The method of claim 16 further comprising the step of placing the graft under tension prior to the preserving step.

29. A method for repairing a damaged cruciate ligament, said method comprising the steps of
preparing a knee of a patient to accept a cruciate ligament replacement graft including drilling a tibial tunnel and a femoral tunnel;
choosing a prepared graft of appropriate size, the prepared graft comprising a length of graft material having a proximal end and a distal end, a first set of sutures attached to the proximal end, and a second set of sutures attached to the distal end, wherein the prepared graft is provided in sterile packaging;
removing the graft from the packaging;
inserting the graft into the prepared femoral and tibial tunnels;
fixing the graft in the femoral tunnel; and
fixing the graft in a tibial tunnel.

30. The method of claim 29 wherein the removing step includes thawing the replacement ligament graft prior to insertion.

31. The method of claim 29 wherein the removing step includes reconstituting the replacement ligament graft prior to insertion.

32. The method of claim 29 wherein the graft further comprises a proximal bone plug.

33. The method of claim 32 wherein the graft further comprises a distal bone plug.

34. The method of claim 29 wherein the fixing steps comprise using fixation devices selected from the group consisting of interference screws, cross-pins, tab-loop anchors, and screws and washers.

35. A kit for replacing a damaged ligament in a patient comprising a sterile packaged prepared replacement ligament having pre-attached sutures for aiding in insertion into the patient, and a graft fixation device.

36. The kit of claim 35 wherein the graft fixation device is selected from the group consisting of interference screws, cross pins, tab-loop anchors, and screws and washers.
